(19) United States
(12) Patent Application Publication
(10) Pub. No.: US 2019/0080785 A1
(43) Pub. Date: Mar. 14, 2019

(54) METHODS OF FORECASTING ENROLLMENT RATE IN CLINICAL TRIAL

(71) Applicant: Gen LI, East Lyme, CT (US)

(72) Inventor: Gen LI, East Lyme, CT (US)

(21) Appl. No.: 16/190,910

(22) Filed: Nov. 14, 2018

Related U.S. Application Data
(63) Continuation-in-part of application No. 16/124,369, filed on Sep. 7, 2018.
Application No. 16/124,369 is a continuation of application No. 14/818,438, filed on Aug. 5, 2015, now abandoned.
(60) Provisional application No. 62/033,844, filed on Aug. 6, 2014.
(60) Provisional application No. 62/694,111, filed on Jul. 5, 2018.

(51) Int. Cl. G16H 10/20; G06F 19/00

(52) U.S. Cl. CPC G16H 10/20; G06F 19/00

(57) ABSTRACT

In one embodiment, the present invention provides a method and system of designing a clinical trial enrollment plan, comprising the use of non-linear regression analysis to model the relationship between a pair of clinical trial parameter, e.g., the relationship between N and GSER (Gross Site Enrollment Rate) and the relationship between N and CTER (clinical trial enrollment rate). The values of the other parameter of the pair can be extrapolated from said regression analysis, wherein said extrapolated values of the parameters are outputted as a design or plan product which will be used in clinical trial for improving the performance

### CROSS-REFERENCE TO RELATED APPLICATIONS

This application is a continuation-in-part of U.S. Ser. No. 16/124,369, filed Sep. 7, 2018, which is a continuation of U.S. Ser. No. 14/818,438, filed Aug. 5, 2015, which claims the benefit of priority of U.S. Ser. No. 62/033,844, filed Aug. 6, 2014. This application also claims the benefit of priority of U.S. Ser. No. 62/694/111, filed Jul. 5, 2018. The entire content and disclosure of the preceding application is incorporated by reference into this application.

### FIELD OF THE INVENTION

This invention relates generally to methods of improving operational effectiveness in clinical trial planning and execution.

### BACKGROUND OF THE INVENTION

To bring new medicines to needy patients faster is a perennial challenge to clinical development organizations around the world. Longer enrollment cycle time, raising development costs, and declining output are some of the challenges in conducting clinical trials. There are limited understandings on the root causes and true drivers behind these struggles.

In general, there are different factors impacting enrollment cycle times. A specifically defined patient population for a particular disease, for example, can impact the ability of trial sites to identify and recruit patients in a defined period of time, thereby impacting the enrollment cycle time. An experienced and successful investigator/site may have better ability to enroll qualified patients compared to an inexperienced investigator/site. Higher portion of experience sites in a pool of sites deployed by a clinical trial can result in shorter enrollment cycle time.

Instinctively, when there are more sites/investigators being deployed for a trial with a defined number of patients needed, one would expect shortened enrollment cycle time. As clinical development organizations are under pressure to deliver new product faster, senior management often is happy to put seemingly “unlimited” resources behind pivotal clinical trials to evaluate promising drug candidates. A simple logic is to add more sites to the pool for enrollment, aiming to “proportionately” shorten enrollment cycle time. However, the goal of proportionally shorten enrollment cycle time is rarely achieved

In another common scenario, when transitioning to a Phase III trial after a successful Phase II trial, people often “extrapolate” the operational results from the Phase II trials(s) to the Phase III trial(s). Using the enrollment rates from the Phase II trial(s) to calculate the number of sites needed for the Phase III trials, one hope to achieve similar enrollment cycle time as what happened in Phase II trial(s). However, the eventual enrollment cycle time is unlikely to be close to the calculation; instead, the enrollment cycle times are generally substantially longer in this situation.

It has been reported that adding extra sites to a clinical trial has only limited impact to enrollment cycle time (1). In order to shorten enrollment cycle time and minimize costs, one usually plans for an optimized number of sites for a clinical trial. However, it is not clear whether there is a pattern between the number of sites deployed in a clinical trial and enrollment cycle time, and whether it is possible to define such pattern in a simple and universally applicable mathematical relationship.

It is widely acknowledged that the determination of the number of investigator sites heavily relies on experience, in particular, the experience of certain individuals, such as physicians, clinical trial administrators. There is no approach yet known for standardizing or weighing different inputs/comments objectively and in a data driven fashion on the value determination of one or more parameters being selected for the clinical trial. In addition, there is no established quantitative relationship to profile the influence if the value adjustment of one or more parameters.

On the other hand, as new drug development by definition is in an innovation business, every new clinical trial being planned and implemented, theoretically, are a scientific and medical adventure. Any lessons from previous clinical trials, if they can be quantified, would reliably and consistently benefit the adventure and facilitate the delivery of new medicines to patients faster and at lower costs.

As shown in left panel of FIG. 1, the conventional approach for clinical trial design has a number of disadvantages including but not limited to:
a) there are very limited samples for reference;
b) there is no baseline that can be established;
c) the improvement cannot be measured;
d) causal analysis cannot be performed in case of failure;
e) the potential influence/risk related to selection of values for certain parameters (or clinical trial plan) in clinical trial are subjective and difficult to be understood by different people/communities; and
f) if certain parameters are implemented for clinical trial, the clinical trial usually expects delays and budget overrun.

The present invention, as typically shown in the right panel of FIG. 1, provides a solution for clinical trial design with the below benefits and/or advantages:
a) Collective learning can be leveraged;
b) An objective baseline can be established;
c) Improvement can be measured;
d) Systematic diagnosis is allowed when trials are in trouble;
e) Timeline and budget can be objectively managed; and
f) the potential influence/risk related to selection of values for certain parameters (or clinical trial plan) in clinical trial are objective and can be easily and effectively understood by different people/communities.

Thus, there is a need to develop new methodology that can quantitatively identify and evaluate the values for clinical trial parameters which can be used to improve clinical trial execution.

### SUMMARY OF THE INVENTION

In one embodiment, the present invention provides a method of collecting/analyzing historical data and designing a clinical trial, by which, once implemented, the operational effectiveness and efficiency of the clinical trial would be improved. The method is based on historical data and information. In one embodiment, the historical data and information include but not limited to one or more clinical trial parameters and their corresponding values related to number of patients, number of investigator site and disease condition of the clinical trial. In one embodiment, the present invention reveals and establishes patterns and mathematical relationships between two of clinical trial parameters, e.g., the relationship between clinical trial enrollment rate (CTER) and the number of investigator sites (N), and the relationship between gross site enrollment rate (GSER) and the number of investigator sites (N). In one embodiment, one of the pair clinical trial parameters is directly obtained from the historical data. In one embodiment, the other parameter of the pair is a parameter calculated by a computing unit based on the historical data. In one embodiment, one parameter of the pair is an expression of the other, e.g., GSER is the number of patients per site per unit period of time, which can be described as GSER=f(N).

In one embodiment, the method of this invention and the quantitative relationship therein define boundaries for clinical trial operational deliverables aligned up with need or priority of the clinical trial. In one embodiment, specific values or ranges of values of one or more clinical trial parameters are easily identified to improve operational deliverables. For example, depending on the need or priority of a particular clinical trial, the clinical trial may be designed or planned for site optimization and cycle time optimization, for example, a relatively small number of sites and/or a relative short enrollment cycle time.

In one embodiment, a number of operational data/information are collected from a number of clinical trials associated with a disease or condition. In one embodiment, the operational data/information include number of investigator sites (N), gross site enrollment rate (GSER, i.e. number of patients per site per unit period of time), and/or clinical trial enrollment rate (CTER, i.e. number of patients enrolled in a defined unit period of time in a trial). In one embodiment, a non-linear regression analysis is conducted to model the relationship among two or more parameters, such as the relationship between N and gross site enrollment rates, and the relationship between N and the clinical trial enrollment rates. In one embodiment, a mathematical function is derived from the non-linear regression analysis. In one embodiment, the mathematical function is a monotonic function. In one embodiment, a graph between a pair of parameters are plotted, for example, a graph of N vs GSER, and a graph of N vs CTER. In one embodiment, the graph further comprises a modeling/fitting curve depicting the relationships between parameters, e.g., the relationship between CTER and the N, and the relationship between GSER and N. In one embodiment, as vast majority of data points in these graphs/charts fall in a narrow band following a definable pattern, the present invention further provides a feasible method, by using these graphs, to forecast the value of one or more parameters such as GSER and/or CTER and other operational deliverables. In one embodiment, the corresponding semi-quantitative or quantitative relationship between parameters is extrapolated from such graph. In one embodiment, the corresponding (semi)quantitative relationship is extrapolated from the graph to profile the relationship between the number of investigator sites and gross site enrollment rate. In another embodiment, a semi-quantitative or quantitative relationship is extrapolated from such graph to profile the correspondence between the number of investigator sites and clinical trial enrollment rate. In one embodiment, the present invention further provides a method, in view of the ‘distance’ to the idea situation, to quantitatively forecast the risk of a potential or proposed value of one or more parameters. In one embodiment, the risk can be expressed as confidence level in percentage, or any other expression indicating the statistical significance.

In one embodiment, the present invention provides a method of designing a clinical trial enrollment plan, comprising the steps of:
(i) collecting clinical trial parameters from a plurality of historical clinical trials associated with a disease or condition, said clinical trial parameters include but are not limited to number of investigator sites (N), gross site enrollment rate (GSER), and clinical trial enrollment rate (CTER), and any other parameters that can be used to evaluate the performance (e.g., effectiveness and efficiency) of the clinical trial;
(ii) conducting non-linear regression analysis to model the relationship between N and GSER, the relationship between N and CTER, or the relationship between N and any other parameter that can be used to evaluate the performance (e.g., effectiveness and efficiency) of the clinical trial, thereby resulting in a mathematical function profiling the quantitative relationship;
(iii) extrapolating from said regression analysis one or more mathematical functions; and
(iv) in view of need or priority of the clinical trial, selecting a value for each of the clinical trial parameters according to the mathematical function, wherein each of the clinical trial parameters with the value is outputted as a design or plan product for the clinical trial associated with the disease or condition. In one embodiment, the gross site enrollment rate (GSER) is defined as the number of patients enrolled at a single investigator site in a unit of time. In one embodiment, the clinical trial enrollment rate (CTER) is defined as the number of patients enrolled in a unit of time.

In one embodiment, an enrollment plan refers to a plan to enroll patients for a clinical trial. In one embodiment, an enrollment plan is designed or created in view of need or priority of the clinical trial. In one embodiment, the need or priority of a clinical trial is one or more expected values for one or more performance parameters. In one embodiment, the performance parameters include but are not limited to budget, GSER, CTER, Site Effectiveness Index (SEI), Adjusted Site Enrollment Rate (ASER), Enrollment Cycle Time (ECT), duration for patient enrollment, site activation, and data collection. In one embodiment, the performance parameters include these parameters above and other parameters that are disclosed and/or defined in the present invention. In one embodiment, the performance parameters also include any other parameter/measurable that can be used to evaluate the performance (e.g., effectiveness and efficiency) of the clinical trial.

In one embodiment, the values of each clinical trial parameter such as gross site enrollment rate in the historical clinical trial are grouped into a set of data bins. Each bin represents a plurality data in which the values of one parameter fall into a range. In one embodiment, the interval of such bin is fixed or predetermined. In one embodiment, the interval of such bin is determined by the system in a manner that a small interval will be selected when there are a large number of available data points. The “binning” of data is designed to help us to see a simple, easy to understand and easier to calculate mathematical relationship. In one embodiment, the “binning” process are carried out by evenly distributing the total of samples (data points associated with clinical trials) into several bins for further analysis. In one embodiment, a pool of data related to 100 trials can be divided into 10 bins in a continuous fashion according to number of investigator sites (N) and each bin includes 10 clinical trials. In one embodiment, when some data points with the values of a parameter, e.g., N., falling in a bin, the bin containing these data points is characterized or labelled by a representative value of the parameters (e.g., N and GSER), e.g., a median value of N and a median value of GSER. Subsequently, a graph of representative value of one parameter (e.g., median value of N) vs representative value of the other parameter (e.g., median value of GSER) is plotted.

In another embodiment, the clinical trial data are grouped into a set of data bins according to the values of clinical trial enrollment rate. In one embodiment, the clinical trial data are grouped into a set of data bins according to the values of two or more parameters. For example, when some data points with the values of N and CTER fall into a given interval of each parameter, these data points are grouped into one bin. In one embodiment, the representative values, e.g., median values, are calculated to represent the bin.

### BRIEF DESCRIPTION OF THE DRAWINGS

FIG. 1 shows a chart comparing the conventional approach (existing art) and a typical approach provided by the present invention.

FIG. 2 shows a chart of Number of Investigator Sites (N) vs Clinical Trial Enrollment Rate (CTER) for clinical trials associated with a single metabolic disease condition with binned data according to one embodiment of the invention.

FIG. 3 shows a chart of N vs CTER for clinical trials associated with a single respiratory disease with binned data according to one embodiment of the invention.

FIG. 4 shows a chart of N vs CTER for clinical trials associated with a single neurologic disease with binned data according to one embodiment of the invention.

FIG. 5 shows the graph of FIG. 2 with binned data fitted by the mathematical function depicting the relationship between CTER and N according to one embodiment of the invention.

FIG. 6 shows the graph of FIG. 3 with binned data fitted by the mathematical function depicting the relationship between CTER and N, according to one embodiment of the invention.

FIG. 7 shows the graph of FIG. 4 with binned data fitted by the mathematical function depicting the relationship between CTER and N, according to one embodiment of the invention

FIG. 8 shows a chart of N vs GSER for clinical trials associated with a single metabolic disease condition with binned data according to one embodiment of the invention.

FIG. 9 shows a chart of N vs GSER for clinical trials associated with a single respiratory disease or condition (left panel), and clinical trials of a single neurologic disease or condition (right panel) with binned data according to one embodiment of the invention.

FIG. 10 shows a chart of Number of Sites (N) vs Gross Site Enrollment Rate (GSER) for clinical trials of a single neurologic disease condition with original data (not binned) according to one embodiment of the invention.

FIG. 11 shows the graph of FIG. 8 with binned data fitted by the mathematical function depicting the relationship between GSER and N according to one embodiment of the invention.

FIG. 12 shows the graph of FIG. 9, left panel, with binned data fitted by the mathematical function depicting the relationship between GSER and N according to one embodiment of the invention.

FIG. 13 shows the graph of FIG. 9, right panel, with binned data fitted by the mathematical function depicting the relationship between GSER and N according to one embodiment of the invention.

FIG. 14 shows a graph with binned data fitted by the mathematical function depicting the relationship between GSER and N and two lines corresponding to confidence level of 95% according to one embodiment of the invention.

FIG. 15 shows a chart of Number of Sites (N) vs Gross Site Enrollment Rate (GSER) for clinical trials associated with chronic obstructive pulmonary disease (COPD) with original data (not binned) according to one embodiment of the invention.

FIG. 16 shows “sweet spots” that optimize enrollment cycle time and N for clinical trials associated with COPD.

FIG. 17 shows a “too much, too late” scenario, in which a large number of sites were added too late to contribute meaningful numbers of patients

FIG. 18 shows a “too few” scenario, in which a long ECT was needed.

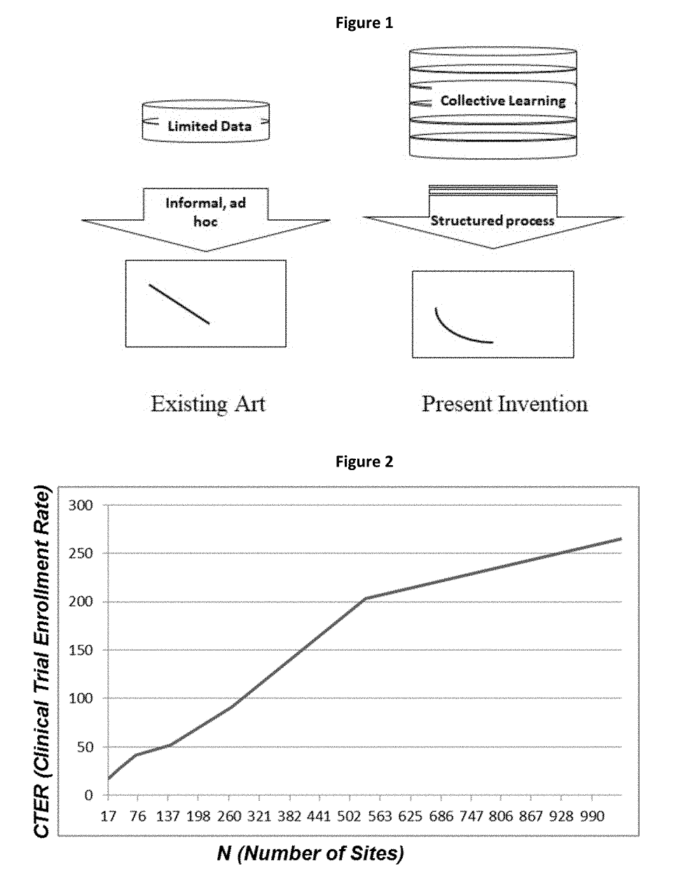

### DETAILED DESCRIPTION OF THE INVENTION

In one embodiment, the present invention provides a method and system of forecasting enrollment rate at clinical trial level (Clinical Trial Enrollment Rate, CTER) and site level (Gross Site Enrollment Rate, GSER), which are a part of clinical trial design and planning.

In one embodiment, Clinical Trial Enrollment Rate (CTER) refers to the number of patients enrolled in a defined unit period of time, e.g. a month, in the duration of clinical trial enrollment period.

In one embodiment, Gross Site Enrollment Rate (GSER) refers to the number of patients enrolled by a single site in a defined unit period of time, e.g. a month, in the duration of clinical trial enrollment period.

In one embodiment, the present invention determines and establishes mathematical relationships between a pair of clinical trial parameters, e.g., between CTER (or GSER) and number of investigator sites (N). The value determination of the clinical trial parameters is important to a comprehensive conceptual framework of the clinical trial design.

In one embodiment, the present invention is featured for use of non-linear regression analysis based on historical clinical data, which leads to a quantitative relationship between a pair of clinical parameters. Not all clinical trials will and can be fitted to the equations disclosed herein perfectly. Imperfect fitting is due to the following:
A targeted age group is too far away from “median” age group;
One or more biochemical and/or physiological and/or genetic measure(s) are too far away from the “median” measures;
Targeted disease status is too far away from a “regular” patient population;
Any other inclusion/exclusion criteria make the planning clinical trial too “unique” due to lack of sufficient historical data.

Since most, if not all, clinical trials will not have a theoretically perfect fit, the present invention provides a practically perfect fitting by using the mathematical models which is highly important and valuable.

In one embodiment, the above list is not exclusive. In one embodiment, the database of the present invention is comprehensive enough to profile vast majority of the impact from these factors in a quantitative way.

In one embodiment, the establishment of quantitative relationship as disclosed in the invention is added to a toolkit as an accessory or an additional function in designing or planning of clinical trial.

In one embodiment, the present invention provides a method and system to define an operational boundary to improve planning of clinical trials. In one embodiment, the sets of mathematical relationships and graphs disclosed in this invention can do more than definition of boundaries. In one embodiment, for a planned clinical trial, when the targeted number of patients is selected, and a desired enrollment cycle time is set, the mathematical equations and/or graphs can be used to optimize the number of sites in view of the need or priority of the clinical trial. This can be to minimize the enrollment cycle time by choosing adequate number of sites; or minimize costs by balancing number of sites and enrollment cycle time.

In one embodiment, upon collection of the data and plotting of a graph, curve fitting can be performed to fit the graph with one or more mathematical formula or functions. Curve fitting is the process of constructing a curve or mathematical function that can best fit a series of data points. For example, curve fitting can involve either interpolation, where an exact fit to the data is required, or smoothing in which a “smooth” function is constructed to approximately fit the data. In one embodiment, curves generated during regression can be used to aid data visualization, by inferring values of a function where no data are available, and by summarizing the relationships among two or more variables.

In one embodiment, the present invention provides a method and system of improving operational effectiveness of a clinical trial implementation, comprising the steps of:
(i) collecting data of clinical trial parameters from a plurality of historical clinical trials associated with a disease condition, said clinical trial parameters comprise number of investigator sites and clinical trial starting dates, clinical trial enrollment ending dates, total number of patients enrolled in a trial, clinical trial design elements, country or countries where the clinical trial is performed, the disease or diseases under investigation by the trial, new disease intervention being tested, and etc.;
(ii) selection of relevant set of clinical trials based on similarity from the parameters mentioned in step (i);
(iii) tabulating the parameters needed for graph plotting, namely Number of Investigator Sites (N), Gross Site Enrollment Rate (GSER), or Clinical Trial Enrollment Rate (CTER);
(iv) plotting a graph of investigator sites vs gross site enrollment rate with original data or binned data;
(v) conducting a non-linear regression analysis of the graph, thereby obtaining a mathematical function that can quantitatively profile the relationship among two or more parameters; and
(vi) in view of need or priority of the clinical trial, extrapolating from said mathematical function a value of one parameter, wherein the parameter includes but is not limited to number of investigator sites, gross site enrollment rate, etc. wherein said extrapolated value of the parameter would improve operational effectiveness of the clinical trial.

In one embodiment, the data points are first binned according to the values of one or more clinical parameters such as investigator sites and gross site enrollment rate. In one embodiment, the bins are characterized by representative values such as median values followed by the non-linear regression analysis.

In one embodiment, the unit of time is one month. In one embodiment, the disease or condition is a metabolic disease, a respiratory disease, a neurologic disease, a cancer and other diseases or conditions that can be studied by randomized clinical trials.

In one embodiment, the mathematical model or function profiling the relationship between investigator sites and gross site enrollment rate is described as: GSER=a·e^{bN}+c, wherein GSER is Gross Site Enrollment Rate; a, b, and c are parameters for a set of clinical trials associated with a disease or condition; b is a negative constant for a set of clinical trials; and N is investigator sites. In one embodiment, the lower limit of site level enrollment rate is c.

In one embodiment, Site Effectiveness Index (SEI) is defined as:

SEI=Σᵢ₌₁^{N}(Etᵢ−Stᵢ)/[(Etₛ−Stₛ)×Nₘₐₓ,

wherein Etᵢ is the date the ith site is closed for patient enrollment (i is a natural number); Stᵢ is the date the ith site is opened for patient enrollment; Nₘₐₓ is maximum number of investigator sites opened for enrollment in the duration of patient enrollment at the study level; Etₛ is the date clinical trial closed for patient enrollment; and Stₛ is the date clinical trial opened for patient enrollment.

In one embodiment, Adjusted Site Enrollment Rate (ASER) is defined as:

ASER=TE/Σᵢ₌₁^{N}(Etᵢ−Stᵢ),

wherein TE is Total Enrollment.

In one embodiment, Nₘₐₓ has a value no bigger than N. In one embodiment, Nmax has a value equal to N. In one embodiment, when Nₘₐₓ equals to N, GSER is related to Site Effectiveness Index (SEI) and Adjusted Site Enrollment Rate (ASER) as: GSER=SEI×ASER,

In another embodiment, the present invention provides a method and system of improving operational effectiveness of a clinical trial, comprising the steps of:
(i) collecting data of clinical trial parameters and their corresponding values from a plurality of historical clinical trials associated with a disease or condition, said clinical trial parameters comprise investigator sites and clinical trial enrollment rate, wherein said clinical trial enrollment rate is defined as the number of patients enrolled in a unit of time;
(ii) conducting a non-linear regression analysis with original or binned data, thereby resulting a mathematical function profiling a quantitative relationship between clinical parameters, wherein said clinical parameters include but are not limited to number of investigator sites vs clinical trial enrollment rate; and
(iii) in view of need or priority of clinical trial, extrapolating value of each clinical trial parameter from said mathematical function, wherein said extrapolated value of said each clinical trial parameter and said mathematical function would lead to accurately define baseline of the picture, identify and implement possible improvements, so to improve operational effectiveness of the clinical trial.

In one embodiment, the values of clinical trial parameters such as investigator sites and clinical trial enrollment rate are first converted into median values before the non-regression analysis.

In one embodiment, the unit of time is one month. In one embodiment, the disease condition is a metabolic disease, a respiratory disease, or a neurologic disease, and other diseases or conditions that can be studied by randomized clinical trials.

In one embodiment, the quantitative relationship between investigator sites (N) and clinical trial enrollment rate (CTER) can be described as: CTER=A·(1−e^{BN})+C, wherein CTER is Clinical Trial Enrollment Rate; A, B, and C are parameters for a set of clinical trials associated with a disease condition; B is a negative constant for a set of clinical trials; and N is investigator sites. In one embodiment, the upper limit of CTER is A+C.

In another embodiment, the present invention provides a non-transitory computer-readable medium with stored instructions. In one embodiment, when executed by a processor, the system performs the steps comprising: (i) collecting from a database clinical trial parameters from a plurality of historical clinical trials associated with a disease or condition, said clinical trial parameters comprise investigator sites, clinical trial enrollment rate, and gross site enrollment rate; (ii) conducting a non-linear analysis, thereby obtaining a mathematical function depicting the quantitative relationship between parameters; and (iii) in view of need or priority of clinical trial, extrapolating values of the clinical parameters from the mathematical function.

In one embodiment, when a desired gross site enrollment rate is known or preferred, the value of investigator sites can be calculated according to the quantitative relationship as described by the mathematical function. In another embodiment, when a desired number of investigator sites is known or preferred, the value of clinical trial enrollment rates can be calculated. In one embodiment, the values of clinical trial parameters such as investigator sites, clinical trial enrollment rate or gross site enrollment rate are first binned before the non-linear regression analysis.

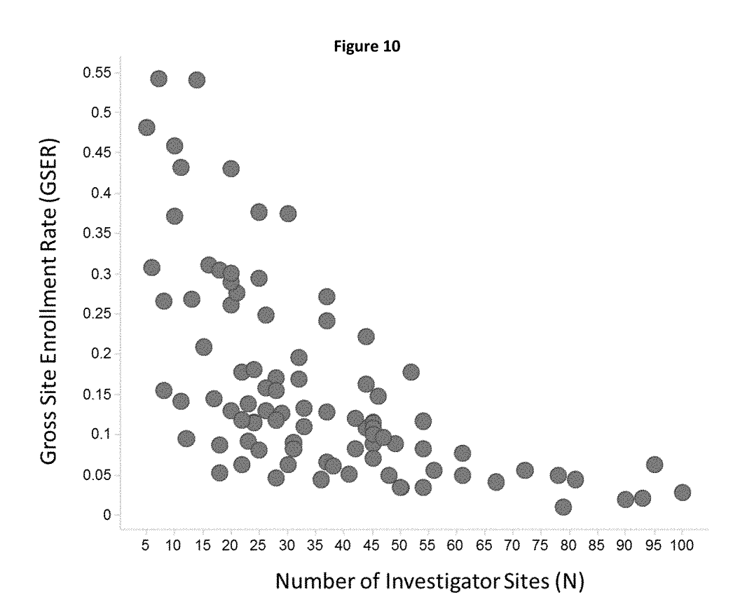

In one embodiment, the above computer-readable medium further comprises an instruction to perform one or more steps of curve fitting after step (ii), i.e., conducting non-linear regression analysis and adding the best fit curve. In one embodiment, as it shown in FIG. 10, the data pattern itself may provide qualitative or semi-quantitative guidance to achieve some results without a non-linear regression and/or the fitting curve.

In another embodiment, the present invention also provides a system for clinical trial planning or design to improve operational effectiveness of a clinical trial, comprising:
(i) a memory for storing a database of clinical trial parameters derived from a plurality of historical clinical trials associated with a disease or condition, wherein said clinical trial parameters comprise number of investigator sites, clinical trial enrollment rate, and gross site enrollment rate; and
(ii) a processor that conducts a non-linear regression analysis between a pair of clinical trial parameters based on historical clinical data, thereby obtaining a mathematical function that describes the quantitative relationship between the pair of clinical trial parameters, wherein said clinical parameters include but are not limited to N, GSER, and CTER,
   wherein the processor, in view of need or priority of the clinical trial, extrapolates the value of the other parameter in the pair, from said quantitative relationship.

In one embodiment, this invention provides a method of designing a clinical trial enrollment plan, wherein said method comprises the steps of:
(i) collecting clinical trial parameters from a plurality of historical clinical trials associated with a disease or condition, wherein said clinical trial parameters comprise number of investigator sites, gross site enrollment rate, clinical trial enrollment rate, any other parameters that can be used to evaluate the performance (e.g., effectiveness and efficiency) of the clinical trial, and any other parameters/measurables that are used to calculate N, GSER, CTER, or other performance parameters;
(ii) conducting a non-linear regression analysis to obtain a mathematical function describing the quantitative relationship between a pair of clinical trial parameters, wherein said clinical trial parameters include number of investigator sites, gross site enrollment rate, clinical trial enrollment rate, any other parameters that can be used to evaluate the performance (e.g., effectiveness and efficiency) of the clinical trial; and
(iii) extrapolating values of the pair of the clinical trial parameters from said quantitative relationship, wherein said extrapolated values of clinical trial parameters are used in one or more clinical trial enrollment plans.

In one embodiment, the non-linear regression analysis for relationship between GSER and N comprises a step of fitting using GSER=a·e^{bN}+c , wherein said GSER is Gross Site Enrollment Rate, e is an exponential function, a, b, c are constants to be determined in the non-linear regression, and N is the number of investigator sites. In another embodiment, c is the lower limit of the gross site enrolment rate. In one embodiment, said GSER is related to Site Effectiveness Index (SEI) and Adjusted Site Enrollment Rate (ASER) as: GSER=SEI×ASER.

In one embodiment, said non-linear regression analysis for relationship between CTER and N comprises a step of fitting using CTER=A·(1−e^{BN})+C , said CTER is Clinical Trial Enrollment Rate, A, B, C are constants to be determined in the non-linear regression, and N is the number of investigator sites. In another embodiment, A+C is the upper limit of the clinical trial enrolment rate.

In one embodiment, the above disease or condition includes but is not limited to a metabolic disease, a respiratory disease, a neurologic disease, and any other diseases or conditions that can be studied by randomized clinical trials.

In one embodiment, this invention further provides a non-transitory computer-readable medium with instructions stored thereon for designing a clinical trial enrollment plan, that when executed by a processor, perform the steps comprising:
(i) collecting from a first database clinical trial parameters from a plurality of historical clinical trials associated with a disease or condition, wherein said clinical trial parameters include but are not limited to N, GSER and CTER;
(ii) conducting non-linear regression analysis to obtain a mathematical function to model the quantitative relationship between a pair of clinical trial parameters; and
(iii) extrapolating values of the pair of clinical parameters from said quantitative relationship, wherein said extrapolated values are used in the design of one or more clinical trial enrollment plans.

In one embodiment, said non-linear regression analysis for relationship between GSER and N comprises a step of fitting using GSER=a·e^{bN}+c, wherein said GSER is Gross Site Enrollment Rate, e is an exponential function, a, b, c are constants to be determined in the non-linear regression, and N is the number of investigator sites. In another embodiment, c is the lower limit of the gross site enrolment rate.

In one embodiment, said non-linear regression analysis for relationship between CTER and N comprises a step of fitting using CTER=A·(1−e^{BN})+C , said CTER is Clinical Trial Enrollment Rate, e is an exponential function, A, B, C are constants to be determined in the non-linear regression, and N is the number of investigator sites. In another embodiment, A+C is the upper limit of CTER.

In one embodiment, the value of N calculated according to the mathematical equation will be rounded or approximated to a natural number (e.g., 1, 2, 3, 4, . . . ), which corresponds to a higher confidence level or a lower risk factor. In one embodiment, the approximated value of N may be smaller than the calculated value. In one embodiment, the approximated value of N may be bigger than the calculated value.

In one embodiment, said disease condition include but are not limited to a metabolic disease, a respiratory disease, and a neurologic disease, or other diseases or conditions studied by randomized clinical trials.

In one embodiment, this invention further provides a system for designing an enrollment plan for a clinical trial associated with a disease or condition, comprising:
(i) a storage unit for storing a database of clinical trial parameters derived from a plurality of historical clinical trials associated with the disease or condition, wherein said clinical trial parameters include but are not limited to N, GSER, CTER, number of patients, age of patient, gender of patient, number of investigator sites, stage or status of one or more diseases, and one or more of biochemical and/or physiological and/or genetic measures;
(ii) a screening unit that selects data in the above database meeting with certain criteria and saves selected data into a sub-database, wherein the criteria are established in view of need or priority of said clinical trial,
(iii) optionally, a grouping unit that bin data or selected data into a set of bins according to one or more intervals, wherein each of said set of bins is characterized by representative values of clinical parameters;
(iv) in view of the need or priority of the clinical trial, one or more processors for conducting non-linear regression analysis based on binned or unbinned data to obtain one or more mathematical functions to model the quantitative relationship between a pair of clinical trial parameters, wherein said one or more processors further extrapolate values of the pair of clinical trial parameters and confidence levels from said mathematical functions;
(v) an outputting unit that outputs the optimal values of the clinical trial parameters that correspond to the highest confidence level,
   wherein said optimal values of the clinical trial parameters are used in the design of one or more clinical trial enrollment plans.

In one embodiment, the present invention provides a method of designing an enrollment plan for a clinical trial associated with a disease or condition, wherein said method comprises the steps of:
(i) collecting data of clinical trial parameters and their values from a plurality of historical clinical trials associated with the disease or condition into a database stored in a storage unit, wherein said data comprise number of investigator sites (N), gross site enrollment rate (GSER), clinical trial enrollment rate (CTER), number of patients, number of investigator sites, stage or status of said disease or condition, and one or more of biochemical, physiological and/or genetic measures, wherein GSER is defined as the number of patients enrolled at one investigator site in a unit of time, and CTER is defined as the number of patients enrolled with a clinical trial in a unit of time;
(ii) screening, via a screening unit, the data in the database meeting with certain criteria, wherein the criteria are established in view of need or priority of said clinical trial;
(iii) optionally grouping the data that meet the criteria into a set of bins according to one or more intervals, wherein each of said set of bins is characterized by representative values;
(iv) on one or more computing units, conducting non-linear regression analysis of the data that meet the criteria, to obtain a mathematical function to model the quantitative relationship between two of the clinical trial parameters,
wherein said non-linear regression analysis for relationship between N and GSER comprises a step of fitting by using GSER=a·e^{bN}+c, wherein a, b and c are constants to be determined in the non-linear regression for said relationship between N and GSER;
wherein said non-linear regression analysis for relationship between N and CTER comprises a step of fitting by using CTER=A·(1−e^{BN})+C, wherein A, B, and C are constants to be determined in the non-linear regression for said relationship between N and CTER;
(v) in view of the need or priority of the clinical trial, through said one or more computing units, extrapolating values for the two of the clinical trial parameters according to said mathematical function, wherein values of a, b and c, and/or A, B and C are determined according to step (iv), extrapolated value of N is or is approximated to a natural number, and confidence levels of a plurality of data points are calculated according to said mathematical function; and
(vi) outputting, via an outputting unit, the optimal values for the two clinical trial parameters for said enrollment plan corresponding to the highest confidence level, wherein said optimal values for said clinical trial parameters are used in designing said enrollment plan for said clinical trial associated with said disease or condition.

In one embodiment, the data that meet the criteria is saved into the database or a sub-database prior to said grouping or non-linear regression analysis.

In one embodiment, the extrapolated value of N is approximated to a natural number which corresponds to a higher confidence level or a lower risk level.

In one embodiment, the confidence levels are mapped out on a chart corresponding to said mathematical function as contour lines representing said confidence levels. In one embodiment, the plurality of data points subject to confidence level calculation includes these data points in view of the need or priority of the clinical trial. In one embodiment, the plurality of data points subject to confidence level calculation includes these data points in view of the need or priority of the clinical trial and all other data points that are necessary for mapping out the contour lines corresponding one or more confidence levels.

In one embodiment, the confidence levels include but are not limited to 70%, 80%, 90%, 95%, 98%, and 99%.

In one embodiment, the disease or condition includes but is not limited to metabolic disease, respiratory disease, neurologic disease, and cancer. In one embodiment, the disease or condition include any other disease or condition that can be studied by randomized clinical trials.

In one embodiment, the quantitative relationship between N and CTER for clinical trials associated with a single metabolic disease or condition is represented by the formula CTER=363.7·(1−e^{-0.00124N})+5.58.

In one embodiment, the quantitative relationship between N and CTER for clinical trials associated with a single respiratory disease or condition is represented by the formula CTER=204.6·(1−e^{-0.00371N})++3.1.

In one embodiment, the quantitative relationship between N and CTER for clinical trials associated with a single neurologic disease or condition is represented by the formula CTER=37.4·(1−e^{-0.0132N}).

In one embodiment, the quantitative relationship between N and GSER for clinical trials associated with a single metabolic disease or condition is represented by the formula: GSER=1.10·e^{-0.0193N}+0.311.

In one embodiment, the quantitative relationship between N and GSER for clinical trials associated with a single respiratory disease or condition is represented by the formula GSER=0.715·e^{-0.00533N}+0.291.

In one embodiment, the quantitative relationship between N and GSER for clinical trials associated with a single neurologic disease or condition is represented by the formula GSER=0.330·e^{-0.00482N}+0.264.

In one embodiment, the present invention provides a system for designing an enrollment plan for a clinical trial associated with a disease or condition, said system comprising:
(i) a memory or storage unit for storing a database of clinical trial parameters and their values derived from a plurality of historical clinical trials associated with said disease or condition, wherein said data include but are not limited to number of investigator sites (N), gross site enrollment rate (GSER), clinical trial enrollment rate (CTER), investigator site, number of enrolled patients, stage or status of one or more diseases, and one or more of biochemical and/or physiological and/or genetic measures, wherein GSER is defined as number of patients enrolled at one investigator site in a unit of time, CTER is defined as number of patients enrolled with a clinical trial in a unit of time;
(ii) a screening unit that selects data in the database meeting with certain criteria, wherein the criteria are established in view of need or priority of said clinical trial;
(iii) optionally, a grouping unit that bins the data that meet the criteria into a set of bins according to one or more intervals, wherein each of said set of bins is characterized by representative values of clinical parameters;
(iv) one or more processors for conducting non-linear regression analysis of the data that meet the criteria, either grouped or ungrouped, to obtain one mathematical function to model the quantitative relationship between two of clinical trial parameters,
   wherein said non-linear regression analysis for relationship between N and GSER comprises a step of fitting by using GSER=a·e^{bN}+c , wherein a, b and c are constants to be determined in the non-linear regression analysis for relationship between N and GSER;
   wherein said non-linear regression analysis for relationship between N and CTER comprises a step of fitting by using CTER=A·(1−e^{BN})+C, wherein A, B, and C are constants to be determined in the non-linear regression analysis for relationship between N and CTER;
   wherein said one or more processors, in view of need or priority of said clinical trial, further extrapolate values of the two of clinical trial parameters and confidence levels of a plurality of data points according to said mathematical function, wherein extrapolated value of N is or is approximated to a natural number; and
(v) an outputting unit, in view of the need or priority, outputting the optimal values for the two clinical trial parameters for said enrollment plan corresponding to the highest confidence level, wherein said optimal values for said clinical trial parameters are used in the design of said enrollment plan for said clinical trial associated with said disease or condition.
   wherein said product or report is used in the design of said enrollment plan for said clinical trial associated with said disease or condition.

In one embodiment, the data that meet the criteria is saved into the database or a sub-database prior to said grouping or non-linear regression analysis. In one embodiment, the certain criteria are established by setting up the minimum or maximum or a range of values for one or more parameter so as to align with the target population of the clinical trial in view of its need or priority. In one embodiment, the criteria are to target a clearly defined population. In one embodiment, the criteria are to target a population of a broader scope.

In one embodiment, the data extrapolated value of N is approximated to a natural number which corresponds to a higher confidence level or a lower risk level.

In one embodiment, the confidence levels are mapped out on a chart corresponding to said mathematical function as contour lines representing said confidence levels. In one embodiment, the plurality of data points subject to confidence level calculation includes these data points in view of the need or priority of the clinical trial. In one embodiment, the plurality of data points subject to confidence level calculation includes these data points in view of the need or priority of the clinical trial and all other data points that are necessary for mapping out the contour lines corresponding one or more confidence levels.

In one embodiment, the confidence levels include but are not limited to 70%, 80%, 90%, 95%, 98%, and 99%.

In one embodiment, the disease or condition includes but is not limited to metabolic disease, respiratory disease, neurologic disease, and cancer. In one embodiment, the disease or condition include any other disease or condition that can be studied by randomized clinical trials.

In one embodiment, the quantitative relationship between N and CTER for clinical trials associated with a single metabolic disease or condition is represented by the formula CTER=363.7 ·(1−e^{-0.00124N})+5.58.

In one embodiment, the quantitative relationship between N and CTER for clinical trials associated with a single respiratory disease or condition is represented by the formula CTER=204.6·(1−e^{-0.00371N})+3.1.

In one embodiment, the quantitative relationship between N and CTER for clinical trials associated with a single neurologic disease or condition is represented by the formula CTER=37.4·(1−e^{-0.0132N}).

In one embodiment, the quantitative relationship between N and GSER for clinical trials associated with a single metabolic disease or condition is represented by the formula GSER=1.10·e^{-0.0193N}+0.311.

In one embodiment, the quantitative relationship between N and GSER for clinical trials associated with a single respiratory disease or condition is represented by the formula GSER=0.715·e^{-0.00482N}+0.291.

In one embodiment, the quantitative relationship between N and GSER for clinical trials associated with a single neurologic disease or condition is represented by the formula GSER=0.330·e^{-0.00482N}+0.264.

In one embodiment, the present invention provides a method of evaluating an enrollment proposal for a clinical trial associated with a disease or condition, wherein said method comprises the steps of:
(i) calculating proposed values of one or more clinical trial parameters in said proposal, wherein said one or more clinical trial parameters include but are not limited to number of investigator sites (N), gross site enrollment rate (GSER), and clinical trial enrollment rate (CTER), wherein GSER is defined as the number of patients enrolled at one investigator site in a unit of time, and CTER rate is defined as the number of patients enrolled in a unit of time;
(ii) projecting a data point corresponding to the proposed values of said one or more clinical trial parameters of said proposal, to a chart corresponding to the mathematical function; and
(iii) via an outputting unit, outputting a value of confidence level for said proposal.

In one embodiment, the present invention provides a for evaluating an enrollment proposal for a clinical trial associated with a disease or condition, said system comprising:
(i) a converting unit that calculates or derives proposed values of one or more clinical trial parameters for said proposal, wherein said one or more clinical trial parameters include but are not limited to number of investigator sites (N), gross site enrollment rate (GSER), and clinical trial enrollment rate (CTER), wherein GSER is defined as the number of patients enrolled at one investigator site in a unit of time, and CTER rate is defined as the number of patients enrolled with a clinical trial in a unit of time;
(ii) a projecting unit for projecting the proposed values of said one or more clinical trial parameters to a chart corresponding to the mathematical function; and
(iv) an outputting unit that outputs a value of confidence level for said proposal.

The invention being generally described, will be more readily understood by reference to the following examples which are included merely for purposes of illustration of certain aspects and embodiments of the present invention, and are not intended to limit the invention.

### EXAMPLE 1

### Relationship Between Clinical Trial Enrollment Rate (CTER) and Investigator Sites

A sub-database of clinical trials meeting the following inclusion criteria was constructed: (i) interventional; (ii) with 10 or more sites; (iii) started in year 2000 or later; and (iv) completed enrollment at the time of analysis. The following trials were excluded: (i) extensional trials; (ii) registration trials; (iii) trials including healthy subjects; and (iv) trials with expanded access. Subsequently, a sub-database of relatively “homogeneous” clinical trials was constructed.

FIG. 2 shows a chart for clinical trials of a single metabolic disease condition. The chart was derived by the following steps:
Selecting trials with a single disease condition as primary condition;
Collecting historical data of selected clinical trial;
Binning the values of one clinical trial parameter, e.g., number of investigator sites into baskets/bins:
   10 to 25 sites
   26 to 50 sites
   51 to 100 sites
   101 to 200 sites
   201 to 400 sites
   401 to 800 sites
   801 to more sites
Calculating the meridian value of CTER for all data falling into the bin;
Optionally building a data table to pair median value of N with median value of CTER, as shown in Table 1;
Outputting the chart with N and CTER as x and y, respectively.

TABLE 1

| Median Sites (N) | Median Trial Enrollment Rate (CTER) |
|---|---|
| 17 | 17.9 |
| 37 | 26.8 |
| 72 | 41.2 |
| 141 | 51.8 |
| 264 | 90.8 |
| 534 | 203 |
| 1047 | 265 |

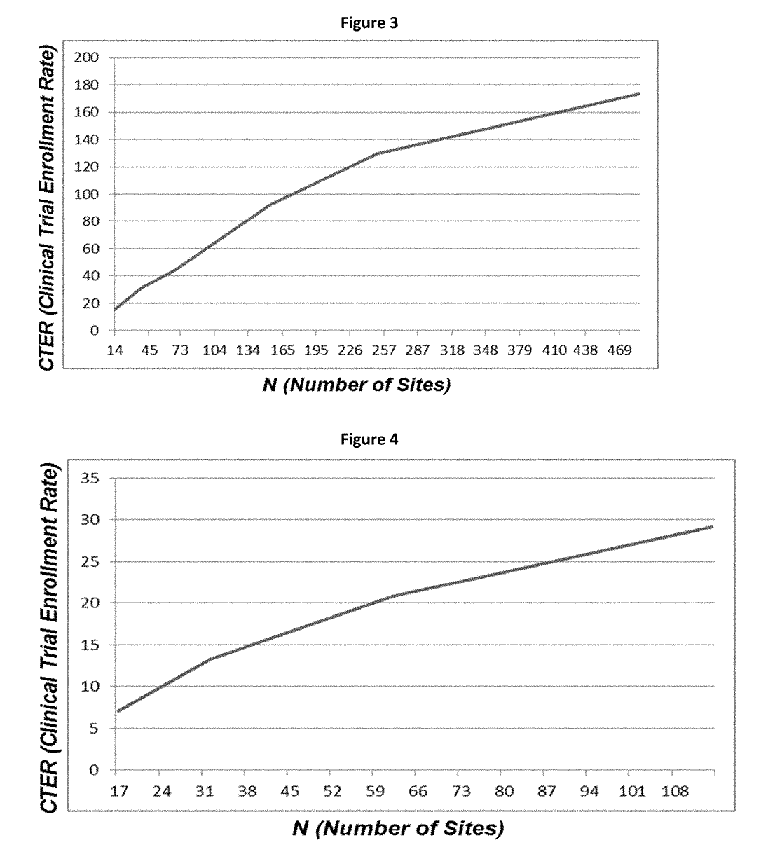

Following these same steps, charts for trials associated with a single respiratory disease and a single neurologic disease were constructed in FIG. 3 and FIG. 4, respectively.

In one embodiment, similar charts can be constructed for a group of clinical trials associated with every single disease or condition, when the sample size is big enough, and the disease or condition is “pure” enough.

As majority of data points in the chart fall in a narrow band, and in definable pattern, it is feasible to use this graph to forecast clinical trial enrollment rate and other operational deliverables.

In each of the charts, as more sites added to a clinical trial in the same disease or condition, the value of CTER increases. The benefit to CTER brought by an addition of every equal number of sites (N), however, diminishes. Eventually, the value of CTER will reach some sort of ceiling, i.e., the benefit from adding numbers of investigator sites becomes negligible.

Thus, there is no “proportionate” relationship between number of sites and clinical trial enrollment rate (CTER). In other words, the relationship between sites and clinical trial enrollment rate are not linear. With an assumption that all other factors are equal or similar, adding sites to a clinical trial can increase CTER, i.e., trial level enrollment rate, and the benefit increment diminishes gradually.

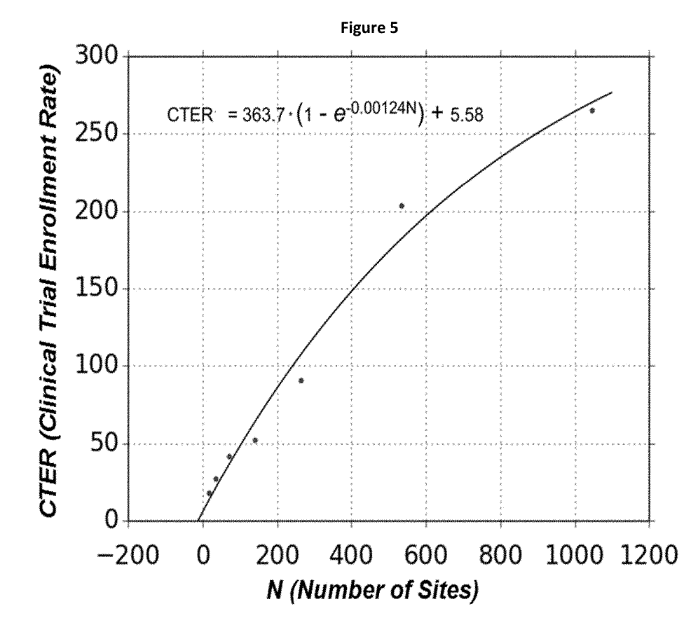

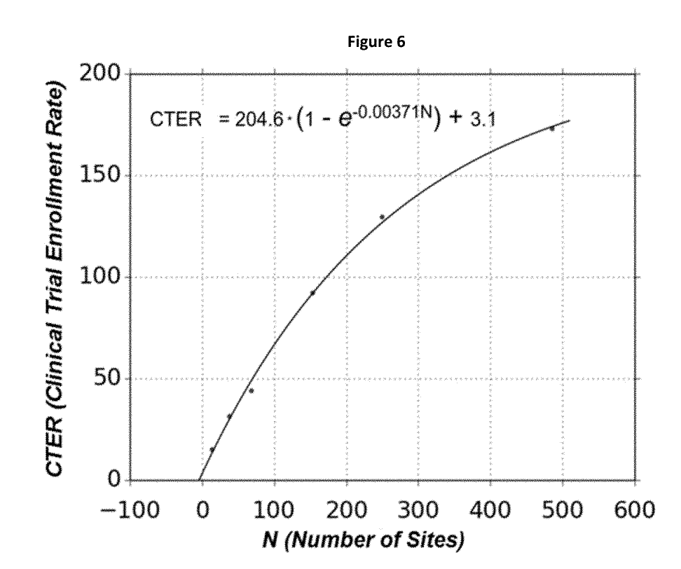

These charts show similar but not identical patterns. A general mathematical function is established to be used for fitting all the charts (see FIGS. 5-7). In one embodiment, the general mathematical function is: CTER=A·(1−e^{BN})+C.

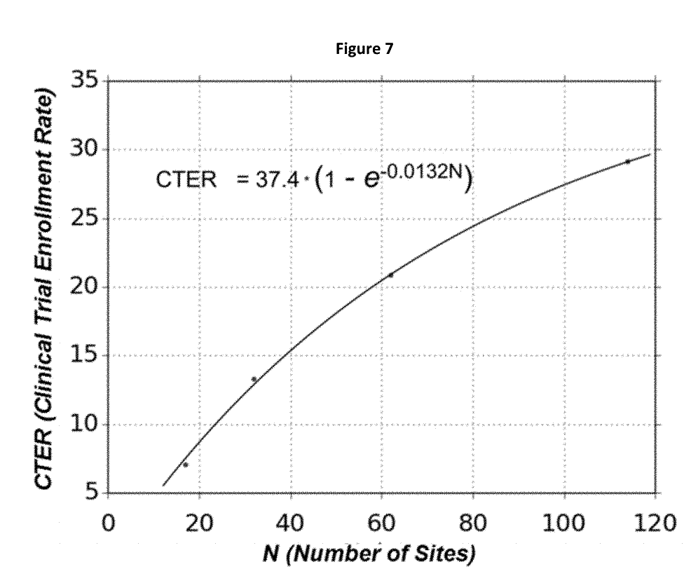

In one embodiment, as shown in FIGS. 4 and 7, the quantitative relationship between N and CTER for clinical trials associated with a single neurologic disease or condition can be described as: CTER=37.4·(1−e^{-0.0132N}).

When the value of CTER is 10 patients per month, the value of N can be calculated as 24 according to the equation. When the value of CTER is 20 patients per month, the value of N can be similarly calculated to be 58. In another words, in order to shorten enrollment cycle time by half through doubling CTER, more than twice as many sites (58 sites instead of 48 sites) would be necessarily added to the pool. This is just an example to illustrate the concept. In practice, it is not usually possible to cut the enrollment cycle time by half.

By now, it is clear that there is an operational boundary for the planning and execution of clinical trials. When more and more sites are added to a clinical trial, the value of CTER will reach a ceiling. Therefore, to shorten enrollment cycle time, addition of investigator sites has a limitation in view of effectiveness and efficiency.

The details of the operational boundaries are being discussed in mathematical terms below. In one embodiment, the following equation describes the relationship between CTER and clinical investigator sites (N): CTER=A·(1−e^{BN})+C.

wherein N is the number of clinical trial investigator sites; e is a mathematical constant; A and B is a negative constant, respectively, for a defined set of clinical trials. In one embodiment, the clinical trial is associated with a single disease or condition.

When N becomes infinitely big, i.e., a very large number of sites is used, the value of e^{BN} becomes close to zero, and the value of CTER will be close to A+C. In other words, no matter how many sites are being deployed in a clinical trial, it is not possible to exceed the trial level of A+C. In reality, one would like to get to as close as possible to A+C, by utilizing as few as possible sites (smaller N). A+C is the upper limit for CTER. Constants A, B, and C are parameters specific to a set of clinical trials associated with a specific and single disease or condition.

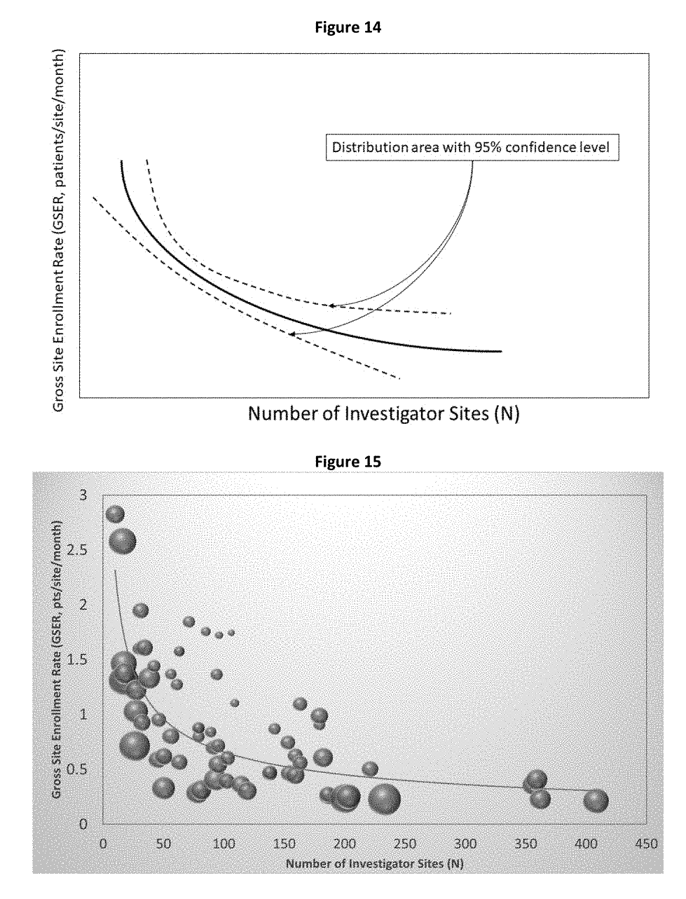

Due to the non-linear nature of the relationship between a pair of parameters, such as the relationship between the enrolment rates and N, it is difficult to accurately obtain the constants a, b, c for GSER or A, B, C for CTER. In one embodiment, in order to overcome this problem, a non-linear regression is employed. In one embodiment, data are modeled by a function in which the parameters are nonlinearly combined. In one embodiment, original or binned data are fitted by a method of successive approximations. The computations for non-linear regression analyses are not feasible without the use of a computer. In one embodiment, the possible values of clinical parameters, such as enrolment rates and number of investigator sites, within a range of the regression model are obtained with certain statistical methods. In one embodiment, a confidence belt can be created/generated from the result of the nonlinear regression within which the enrolment rates and number of investigator sites are expected to be achievable at a certain risk or confident level, e.g., 95%, as typically shown in FIG. 14. In one embodiment, the risk or confident level is measured in view of the distance to the curve corresponding to the ideal mathematical function. In one embodiment, the risk or confident level is expressed in a manner that indicates statistical significance. In one embodiment, the present invention provides a chart comprising a fitted curve corresponding to the quantitative relationship and one or more contour lines corresponding to various levels of confidence.

In one embodiment, the present invention provides a method and system of evaluating a proposed plan with one or more parameters. The method comprises the steps of
(i) generating a chart of a pair of clinical trial parameters based on historical clinical data, wherein said chart comprises one or more contour lines corresponding to various levels of confidence;
(ii) calculating or obtaining a data point with values of the pair of clinical trial parameter in the proposed plan;
(iii) projecting the data point of the proposed plan onto the chart; and
(iv) outputting a result comprising the chart and the data point.

In one embodiment, segmented regression can also be used if the data cannot be fitted into a single model.

### EXAMPLE 2

### Relationship Between Gross Site Enrollment Rate (GSER) and Investigator Sites

Using the same approach as discussed in Example 1, site level enrollment rate (GSER, Gross Site Enrollment Rate) was investigated. Starting from the same sub-database as being used to understand CTER, the chart between N and GSER was derived by the following steps:
Selecting trials with a single disease or condition as primary condition;
Collecting historical data of selected clinical trial;
Binning the values of one clinical trial parameter, e.g., the number of investigator site:
   10 to 25 sites
   26 to 50 sites
   51 to 100 sites
   101 to 200 sites
   201 to 400 sites
   401 to 800 sites
   801 to more sites
Calculating the meridian value of GSER for all data falling into the bin;
Optionally building a data table showing median values of number of sites and GSER) (Table 2);
Outputting the chart with N and GSER as x and y, respectively

TABLE 2

| Median Sites (N) | Median Site Enrollment Rate (GSER) |
|---|---|
| 17 | 1.13 |
| 37 | 0.79 |
| 72 | 0.6 |
| 141.5 | 0.43 |
| 264.5 | 0.3 |
| 534 | 0.31 |
| 1047 | 0.29 |

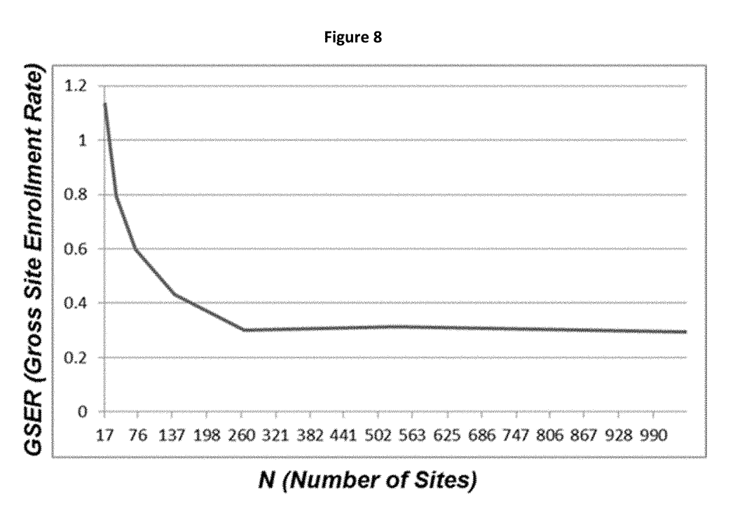

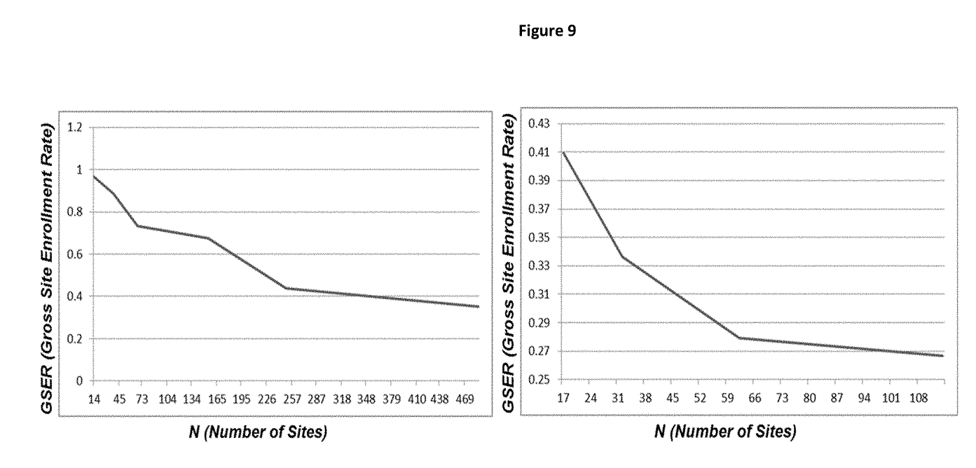

The charts shown in FIGS. 8-9 have different sizes and shapes. The pattern, however, is similar: as the value of N in a set of clinical trials associated with a single disease or condition increases, the value of GSER decreases. It is not linearly correlated. Rather, GSER drops more quickly when the clinical trials involve smaller number of sites. It stabilizes at a certain level when the clinical trials become big enough.

As majority of data points in the chart fall in a narrow band, and in definable pattern, it is feasible to use this graph to forecast gross site enrollment rate and other operational deliverables.

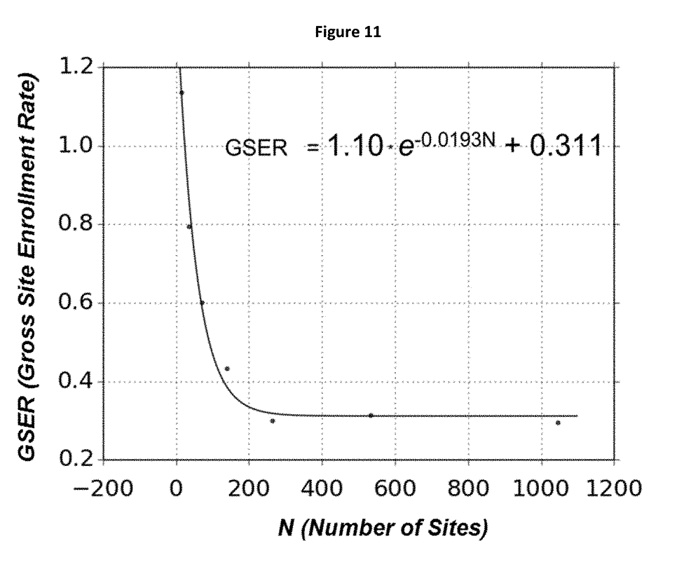

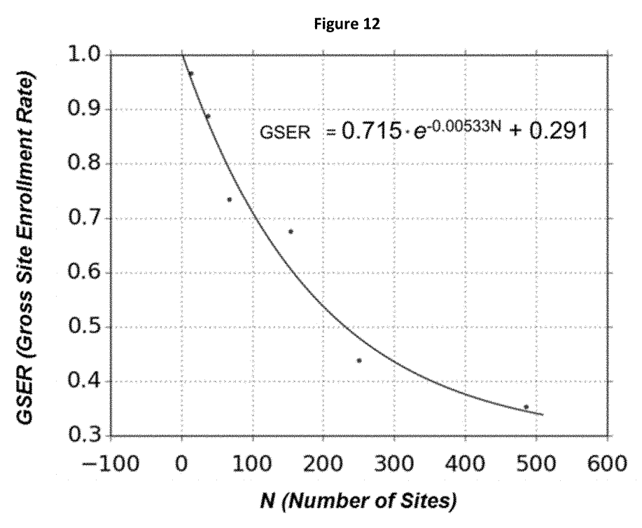

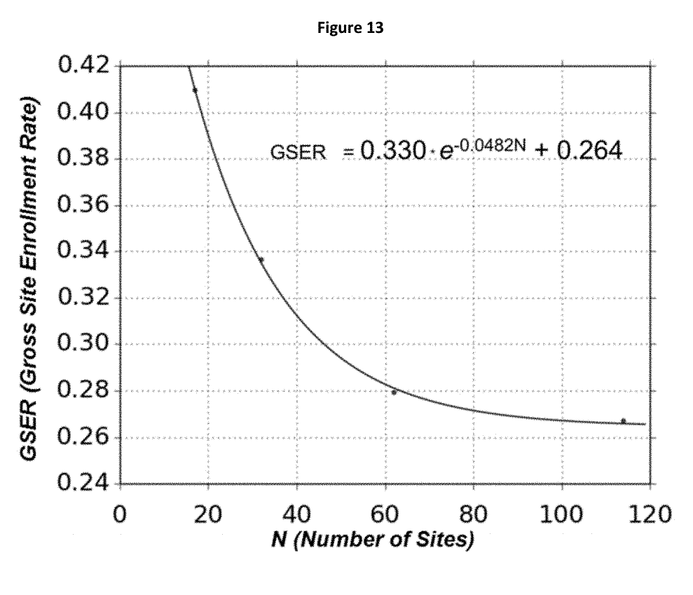

In one embodiment, mathematical relationships profiling the pattern can be shown in FIGS. 11-13. In one embodiment, the mathematical relationships can be represented by the following equation:

GSER=a·e^{bN}+c,

wherein b is a negative constant for a defined set of clinical trials (usually a single disease condition). When the value of N becomes infinitely large (use of very large number of sites), the value of e^{bN} becomes next to zero, and GSER will become close to c. That is to say, the value of GSER cannot be smaller than c. The farther away one can stay from c by reducing the number of sites deployed in a clinical trial, the more one will be able to improve collective site enrollment performance in a clinical trial. In other word, c is the lower boundary for site level enrollment rate. Constants a, b, and c are parameters specific to a set of clinical trials associated with a specific and single disease or condition.

As discussed above, when transitioning to a Phase III trial after a successful Phase II trial, the value of GSER in Phase II trial cannot be simply applied to Phase III because Phase II is usually smaller than Phase III. The value of GSER of Phase II is usually larger than that in Phase III. Accordingly, when one tries to extrapolate the potential values of clinical trial parameters for a Phase III clinical trial, based on historical data from s from a plurality of Phase II clinical trials, e.g., using the Phase II's GSER data to predict the enrollment cycle time for a planning Phase III trial, one may likely end up with disappointing results, which may lead the Phase III clinical trial with a longer enrollment cycle time and frequently followed by a “rescue mission”.

Many factors can be used to explain why larger trials have a lower GSER than those of smaller trials. It has been previously established that the enrollment performance for a pool of sites deployed in a clinical trial can be as measured by Adjusted Site Enrollment Rate (ASER, number of patients per site per month). ASER is impacted by the effectiveness of site activation process, which is measured by Site Effectiveness Index (SEI, 0%<SEI<100%). In one embodiment, with the introduction of GSER, a simple formula can be used to link them together:

GSER=ASER×SEI.

Site Effectiveness Index (SEI) and Adjusted Site Enrollment Rate (ASER) have been defined as (2, 3):

SEI=Σᵢ₌₁^{N}(Etᵢ−Stᵢ)/[(Et_{S}−St_{S})×Nₘₐₓ],

wherein Etᵢ is the time (date) site i closed for patient enrollment; Stᵢ is the time (date) site i opened for patient enrollment; Nₘₐₓ is maximum number of sites opened for enrollment in the duration of patient enrollment at the study level; Etₛ is the time (date) clinical study (trial) closed for patient enrollment; Stₛ is the time (date) clinical study (trial) opened for patient enrollment.

ASER=TE/Σᵢ₌₁^{N}(Etᵢ−Stᵢ),

wherein, TE is Total Enrollment.

In one embodiment, when it is in the planning stage, TE is targeted patient enrollment. In one embodiment, when historical data are being evaluated, TE is the actual number of patients enrolled in a clinical trial.

The above relationships have been tested and have provided superior site enrollment results consistently (4).

As more sites (N) are involved in a clinical trial, operational complexity increases, leading to the decrease of SEI. In return, GSER will be reduced. It is always difficult to find investigator sites with high performance. It becomes even more difficult when a larger number of sites need to be identified. It is not surprising that the average enrollment performance for a trial with a large number of sites is lower than these of trials with a small number of sites.

In one embodiment, the present invention would level the playground for stakeholders in clinical trial planning and execution. In one embodiment, the present invention improves the effectiveness of communication among stakeholders. In one embodiment, the present invention objectively rewards colleagues to achieve quantifiable improvements, and provides actionable opportunities to improve operational deliverables through better site selection, better process, etc. In one embodiment, the establishment of a reliable way to forecast enrollment rate, both at clinical trial level (CTER), and at site level (GSER), will greatly enhance our ability to achieve these objectives. In one embodiment, the present invention also provides a method and system to evaluate a hypothetical clinical trial plan according to the established relationship and/or mathematical functions.

### EXAMPLE 3

### Determining Gross Site Enrollment Rate (GSER)

In planning an oncology clinical trial, a plan proposes using 150 sites to enroll 189 patients in 21 months.

In order to determine whether the above parameters are practical and feasible for the clinical trial, clinical trial parameters such as enrollment cycle time, number of patients enrolled, and number of investigator sites were first collected from clinical trials associated with the same or similar oncology indication. In one embodiment, clinical trials with a total enrollment of between 100 and 300 patients were chosen to ensure similar operational complexity of the trial in planning. In one embodiment, Gross Site Enrollment Rate (GSER) is calculated with the following formula: GSER=number of patients enrolled/number of sites/enrollment cycle time according to these historical data.

Next, a chart with N and GSER as x and y, respectively, is created as shown in FIG. 10. This chart depicts a clear pattern between N and GSER, and this chart can be used to examine various scenarios for clinical trial planning. For example, in one embodiment, one may pick 45 as the value for N and determine from this chart that the corresponding value for GSER. In one embodiment, with or without curve fitting, when the value of N is 45, the value of GSER of ˜0.15 patients per site per month may be extrapolated from this chart. This scenario falls inside the established pattern depicted by the chart, indicating that some previous trials with identical or similar setting have been successfully planned and executed, and that it is reasonable to expect that the planned trial is likely to succeed in enrolling the desired number of patients within the expected time constraint at a certain level of confidence or risk.

In another embodiment, the value for N is selected as 70, the corresponding value of GSER can be determined from this chart. In one embodiment, with or without curve fitting or non-linear regression analysis, the value of GSER of about 0.12 patients per site per month can be extrapolated from this chart. This scenario falls off the established pattern depicted by the chart. In one embodiment, while there is no historical data to support the pair of these parameters (i.e., N=70 and GSER=0.12), since this scenario is very close to the established pattern, one may expect that a clinical trial using these two parameters would have a reasonably good chance to be successfully executed.

In yet another embodiment, one may determine from this chart the corresponding value of GSER may be determined from this chart when the value of N is 150. As shown in FIG. 10, N=150 falls far away outside the established pattern depicted by the chart. Selection of a point falling outside the pattern would lead to a clinical trial with GSER near zero, indicating that a unreasonably long enrollment cycle time is expected and it is not feasible to have a trail with a N's value as 150.

### EXAMPLE 4

### Determination of number of investigator sites (N) and Enrollment Cycle Time (ECT)

The pharmaceutical research landscape is littered with the remains of failed clinical trials. Since 2008, 17.2% of Phase 2 trials and 12.2% of Phase 3 trials have been prematurely terminated, according to an analysis of more than 320,000 clinical trials and over 500,000 investigators across several hundred disease indications. Given that estimated global pharmaceutical R&D spending currently amounts to $125-$160 billion annually,^{5,6} those terminations mean roughly $20 billion is essentially wasted every year. More importantly, terminated trials dash the hopes of patients who could have potentially benefitted from the medical innovations that might have emerged from successful trials.

The above analysis also reveals that patient recruitment difficulties are responsible for 57% and 54% of the failure of Phase 2 Phase 3 trials, respectively. These difficulties result from a variety of factors including suboptimal protocol design, inefficient business processes (especially with regard to site activation), and poor investigator site performance. These difficulties are avoidable and can be addressed through better understanding of the operational characteristics of clinical trials, which itself can lead to improved clinical trial planning.

At the risk of oversimplification, safety and/or efficacy data are collected from a well-defined group of patients for analysis in a highly regulated and carefully controlled setting. Depending on how a variable is defined, it usually takes several dozens or even hundreds of variables to determine the outcomes of a clinical trial. However, due to lack of objective and quantitative approach, even when a trial sponsor (or the CRO it works with) does a hundred things right, one mistake can jeopardize a trial's success.

Oftentimes, success may hinge on the trial planner's appreciation of the complexity of the disease, or on a team's ability to determine the appropriate number of patients, the right number of investigator sites, and the optimal duration of the trial. While each of these factors is a major driver of clinical trial costs, the numbers of patients and sites typically generate relatively little discussion from a financial perspective. Moreover, the clinical trial process is idiosyncratic, dependent on variable experience, and usually conducted without regard to the broader experience of similar trials that have already taken place.

To a great extent, the inattention given to these factors stems from simplistic, perhaps wishful planning and unrealistic, uncalibrated expectations: pharmaceutical companies generally want to get their new medicines to patients as soon as possible and at the lowest possible cost.

The desire for speed can encourage a risky form of linear thinking: for many Phase 3 trials, the operational model for Phase 3 trial can be derived from a successful Phase 2 trial, from which the number of investigator sites is extrapolated in order to attain a similar enrollment cycle time (ECT), which is the elapsed time from first to last enrolled patient, as shown in the following hypothetical example of a clinical program for an investigational anticancer agent.

TABLE 3
Planned versus actual patient enrollment metrics
for a Phase 3 oncology clinical trial

| | Phase 2 -- | Phase 3 -- | Phase 3 -- |
|---|---|---|---|
| | actual | planned | actual |
| Patients | 160 | 970 | 970 |
| Sites | 48 | 280 | 258 |
| Enrollment cycle time (ECT) | 14 | 12 | 24 |
| (months) | | | |
| Gross site enrollment rate (GSER) | 0.29 | 0.29 | 0.15 |
| (patients/site/month) | | | |
| Site effectiveness index (SEI) | 0.68 | unknown | 0.71 |

In the above example, the trial planners assumed a linear relationship between number of patients, number of sites, and ECT and, by extrapolating the ECT value of 14 months in the Phase 2 trial, forecasted an ECT value of 12 months for the Phase 3 trial. Unfortunately, such assumption of the linear relationship is not applicable: the actual ECT value for the Phase 3 trial was 24 months—twice of the forecast. A Phase 3 trial is not merely a bigger Phase 2 trial; oftentimes, this is a costly lesson.

The present invention provides a predictive analytics platform that consolidates comparable trial and site metrics to support trial design, protocol design, site selection, and trial execution. Although no two trials are exactly alike, the platform yields a mathematical relationship that enables a “comparison of the incomparables” using the following metrics, together with other metrics disclosed in the present invention^{2,7,8}.
Site Activation Curve: the number of sites activated over time
Enrollment Curve: the number of patients enrolled over time

It seems intuitive to add sites to a trial in order to have them contribute more patients and thereby reduce ECT. What is less intuitive, however, is that the incremental benefit vanishes at a certain point, beyond which the ECT is prolonged. As shown in FIG. 15, the declining GSER means each site contributes fewer patients over a defined period of time (ECT). In other words, the point of diminishing returns is reached early in the course of the trial, in part because of slow site activation (a particularly thorny problem for large studies with many sites), in part because the best sites are recruited first. Late activation of a poorly performing site pulls down the site activation curve. This distinctive pattern holds true for over 1,000 different disease indications analyzed according to the approach provided by the present invention. It can be used to any other disease or condition according to the knowledge and understanding of a skilled person in the art.

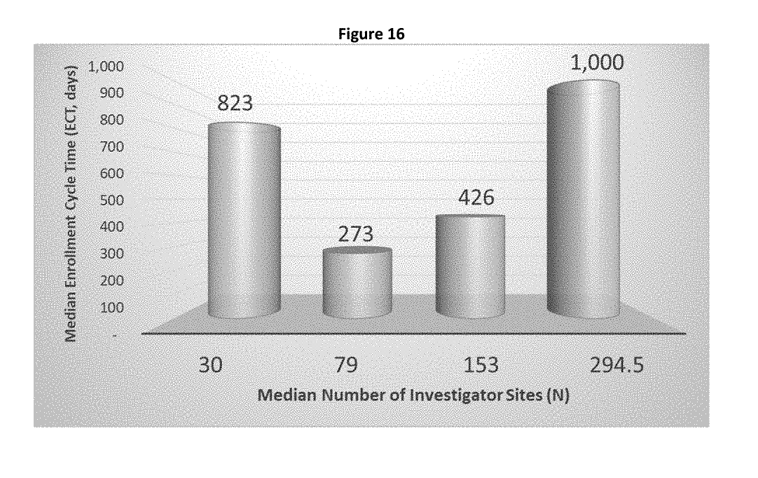

FIG. 16 further pinpoints the optimized scenario at the point where activating 79 sites would yield an ECT of 273 days. Beyond this boundary, the benefits diminish. As shown in FIG. 16, the enrollment and site activation patterns, coupled with the observed mathematical relationships, essentially enable us to objectively determine the optimal number of investigator sites (alternatively number of sites). Moreover, the predictive analytics platform facilitates the optimization of clinical trial design and country-to-country comparison of site performance, among many other possibilities.

### Too Many Sites

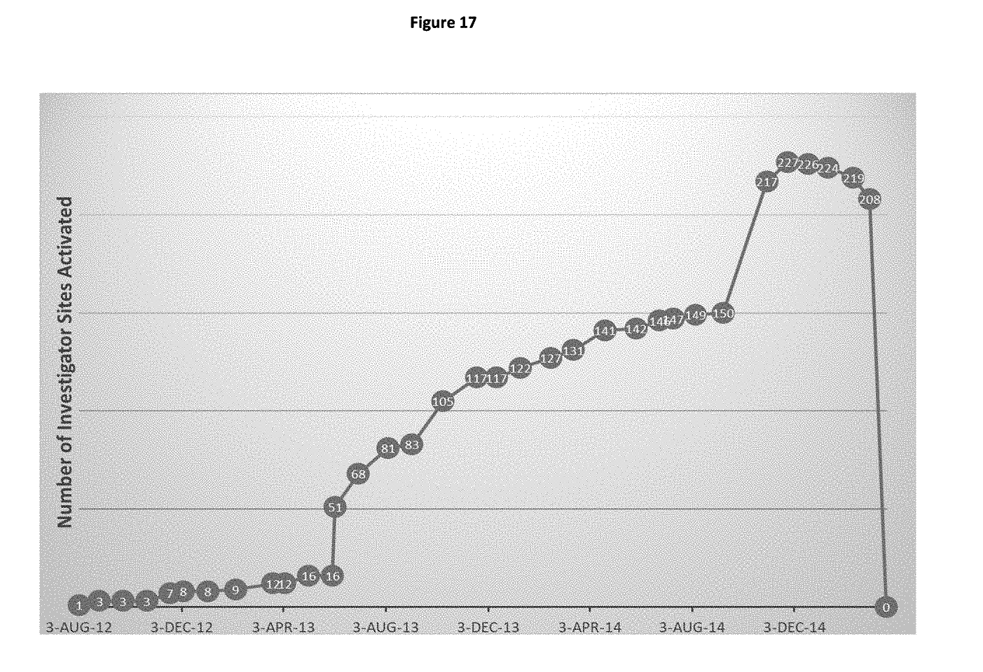

One might argue that even if the GSER decreased, there would still be a surplus of eligible patients to potentially reduce the ECT. But that is not what we get in reality (see FIG. 17).

Why do the benefits fall off so dramatically? It's because activating an excessive number of investigator sites yields a larger trunk of non-performing sites that drain financial resources and, in all likelihood, prolong the ECT. In the example illustrated in FIG. 17, a total of 227 sites were activated in this trial, but only about 140 sites contributed patients. Moreover, the 77 sites activated in the last six months of the trial did not contribute a meaningful number of patients. The number of activated sites far exceeded the 120 sites recommended via our optimization analysis, as illustrated in FIGS. 15 and 16. Additionally, the 87 non-performing sites created a financial exposure amounting to $10.4 million, based on an assumed $30,000 in site activation costs and $3,000 per site per month over a 30-month duration. Those costs yielded an SEI of 44%, significantly lower than the recommended 60% SEI value for this trial.⁹

The disparity between actual and recommended SEI illustrates one of the perils of activating too many investigator sites: activating a large number of sites takes time, especially in the early stages of a trial. In the trial described above, the team was forced to push too many sites forward with limited resources, and a large percentage of sites were activated near the end of the ECT, when the team was spread too thin by focusing on too many nonproductive tasks, and was unable to focus on maximizing returns from the most productive sites. In short, there is such a thing as “too big to succeed.”

### Too Few Sites

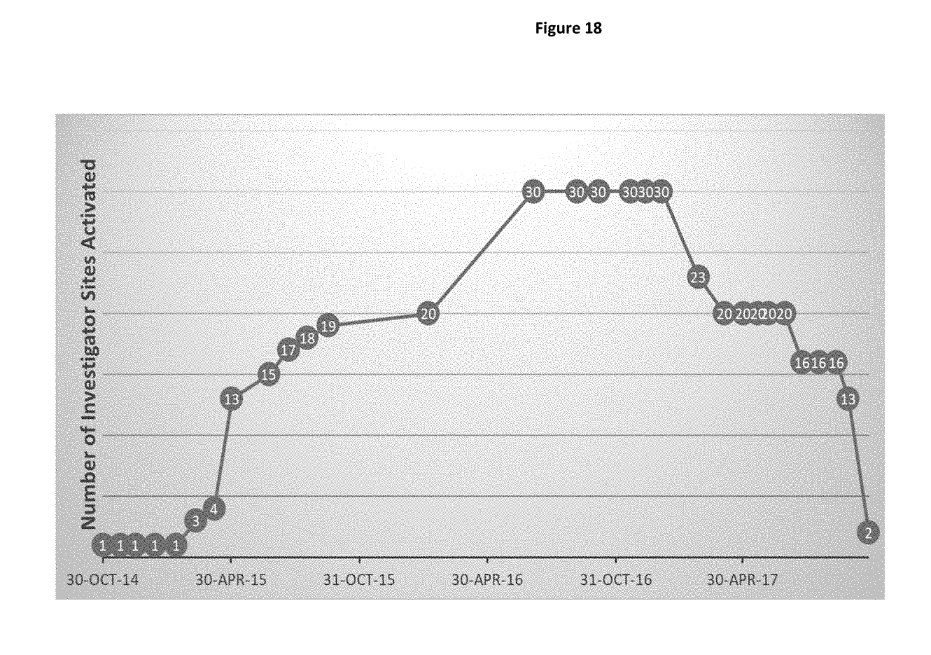

Moving in the opposite direction risks crossing another boundary, one that results from activating too few sites rather than too many. Such a situation may occur when a budget-conscious sponsor funds an insufficient number of sites (see FIG. 18).

In this case, analysis according to the method provided in the present invention yielded a recommendation of 80 sites and a forecasted ECT of 15 months. The trial team, restricted by available funding, decided to activate 30 sites instead. The lower number of sites reduced site activation costs by about $1.5 million. The trial team used these savings to extend site management over a much longer time frame, from 15 months to 35 months. Unfortunately, the savings were negated by extra costs for drug supply, medical monitoring, and various other project management costs. The 20 extra months in ECT therefore constituted wasted time and a lost opportunity to optimize the site activation timeline. Presumably, advance knowledge of these opportunity costs would have prompted management to make a different decision about this trial.

### Enhancing Organizational Awareness of the Boundary

In one embodiment, the present invention provides an analytics platform which is objective and quantitative and enables trial planning and execution in an integrated fashion. Nevertheless, true integration is not a given. In many big pharma companies, and even in some small ones, siloed decision-makers can jeopardize clinical trial success. Even if the trial planner is aware of the point of diminishing returns (and of the risks of disregarding this critical juncture), this knowledge is irrelevant unless it is shared across the organization. That speaks to the importance of cross-functional communication between the medical, clinical, commercial, regulatory, and finance teams—as well as between sponsor and CRO—to optimize decision-making. When each of these parties understands the importance of the factors that affect site activation and patient enrollment, and of the variables that determine enrollment rates and site performance, the organization as a whole (and its CRO partner) can successfully navigate what might otherwise be a perilous clinical trial landscape.

### REFERENCES

(1) Gen Li, Lauri Sirabella, 2010. Planning the Right Number of Investigative Sites for a Clinical Trial. The Monitor 2010; 24(4): 54-58.

(2) Gen Li and Beth Harper, 2009. Finding the Sweet Spot. PharmExec Oct. 2, 2009. http://www.pharmexec.com/finding-sweet-spot.

(3) U.S. Pat. No. 8,271,296

(4) Robert Gray, Gen Li, 2011. Performance-Based Site Selection Reduces Costs and Shortens Enrollment Time. The Monitor 2011; 25(7): 32-36.

(5) ICON plc presentation at 35^{th} Annual J. P. Morgan Healthcare Conference, San Francisco, Calif., 2017 Jan. 10. http://files.shareholder.com/downloads/ICLR/3717560502x0x923636/3ECDE4CD-8149-47D3-86B0-F81B69616D25/ICON JPM 2017 10 Jan. Final.pdf.

(6) Catalent, Inc. presentation at 34^{th} Annual J. P. Morgan Healthcare Conference, San Francisco, Calif., 2016 Jan. 11. http ://investor.catalent.com/sites/catalent.investorhq.businesswire.com/files/event/additiona1/2 016.01.08—CTLT—JPM HC Conference FINAL.pdf

(7) Li G. Site activation: the key to more efficient clinical trials. Pharmaceutical Executive. 2008 Dec. 12. http://www.pharmexec.com/site-activation-key-more-efficient-clinical-trials.

(8) Li G. Forecast enrollment rate in clinical trials. Applied Clinical Trials. 2015; 35(3):42-48. http://www.appliedclinicaltrialsonline.com/forecast-enrollment-rate-clinical-trials-0.

(9) Legagneur V, Peachey J, Correa K, Li G. Enrollment cycle times can and should be optimized. Applied Clinical Trials. 2018 Jan. 17. http://www.appliedclinicaltrialsonline.com/enrollment-cycle-times-can-and-should-be-optimized.

What is claimed is:

1. A method of designing an enrollment plan for a clinical trial associated with a disease or condition, wherein said method comprises the steps of:
(i) collecting data of clinical trial parameters and their values from a plurality of historical clinical trials associated with the disease or condition into a database stored in a storage unit, wherein said data comprise number of investigator sites (N), gross site enrollment rate (GSER), clinical trial enrollment rate (CTER), number of patients, number of investigator sites, stage or status of said disease or condition, and one or more of biochemical, physiological and/or genetic measures, wherein GSER is defined as the number of patients enrolled at one investigator site in a unit of time, and CTER is defined as the number of patients enrolled with a clinical trial in a unit of time;
(ii) screening, via a screening unit, the data in the database meeting with certain criteria, wherein the criteria are established in view of need or priority of said clinical trial;
(iii) optionally grouping the data that meet the criteria into a set of bins according to one or more intervals, wherein each of said set of bins is characterized by representative values;
(iv) on one or more computing units, conducting non-linear regression analysis of the data that meet the criteria, to obtain a mathematical function to model the quantitative relationship between two of the clinical trial parameters,
wherein said non-linear regression analysis for relationship between N and GSER comprises a step of fitting by using GSER=a·e^{bN}+c, wherein a, b and c are constants to be determined in the non-linear regression for said relationship between N and GSER;
wherein said non-linear regression analysis for relationship between N and CTER comprises a step of fitting by using CTER=A·(1−e^{BN})+C, wherein A, B, and C are constants to be determined in the non-linear regression for said relationship between N and CTER;
(v) in view of said need or priority of the clinical trial, through said one or more computing units, extrapolating values for the two of the clinical trial parameters according to said mathematical function, wherein values of a, b and c, and/or A, B and C are determined according to step (iv), extrapolated value of N is or is approximated to a natural number, and confidence levels of a plurality of data points are calculated according to said mathematical function; and
(vi) outputting, via an outputting unit, the optimal values for the two clinical trial parameters for said enrollment plan corresponding to the highest confidence level, wherein said optimal values for said clinical trial parameters are used in designing said enrollment plan for said clinical trial associated with said disease or condition.

2. The method of claim 1, wherein said confidence levels are mapped out on a chart corresponding to said mathematical function as contour lines representing said confidence levels.

3. The method of claim 1, wherein said extrapolated value of N is approximated to a natural number which corresponds to a higher confidence level.

4. The method of claim 1, wherein said confidence levels are selected from the group consisting of 70%, 80%, 90%, 95%, 98%, and 99%.

5. The method of claim 1, wherein said disease or condition is selected from the group consisting of metabolic disease, respiratory disease, neurologic disease, and cancer.

6. The method of claim 1, wherein the quantitative relationship between N and CTER for clinical trials associated with a single metabolic disease or condition is represented by the formula CTER=363.7·(1−e^{-0.00124N})+5.58.

7. The method of claim 1, wherein the quantitative relationship between N and CTER for clinical trials associated with a single respiratory disease or condition is represented by the formula CTER=204.6·(1−e^{-0.00371N})+3.1.

8. The method of claim 1, wherein the quantitative relationship between N and CTER for clinical trials associated with a single neurologic disease or condition is represented by the formula CTER=37.4·(1−e^{-0.0132N}).

9. The method of claim 1, wherein the quantitative relationship between N and GSER for clinical trials associated with a single metabolic disease or condition is represented by the formula: GSER=1.10·e^{-0.0193N}+0.311.

10. The method of claim 1, wherein the quantitative relationship between N and GSER for clinical trials associated with a single respiratory disease or condition is represented by the formula GSER=0.715·e^{-0.00533N}+0.291.

11. The method of claim 1, wherein the quantitative relationship between N and GSER for clinical trials associated with a single neurologic disease or condition is represented by the formula GSER=0.330·e^{-0.00482N}+0.264.

12. A system for designing an enrollment plan for a clinical trial associated with a disease or condition, said system comprising:
(i) a memory or storage unit for storing a database of clinical trial parameters and their values derived from a plurality of historical clinical trials associated with said disease or condition, wherein said data comprise the group consisting of number of investigator sites (N), gross site enrollment rate (GSER), clinical trial enrollment rate (CTER), number of investigator sites, stage or status of said disease or condition, and one or more of biochemical, physiological and/or genetic measures, wherein GSER is defined as number of patients enrolled at one investigator site in a unit of time, CTER is defined as number of patients enrolled with a clinical trial in a unit of time;
(ii) a screening unit that selects data in the database meeting with certain criteria, wherein the criteria are established in view of need or priority of said clinical trial;
(iii) optionally, a grouping unit that bins the data that meet the criteria into a set of bins according to one or more intervals, wherein each of said set of bins is characterized by representative values of clinical parameters;
(iv) one or more processors for conducting non-linear regression analysis of the data that meet the criteria, either grouped or ungrouped, to obtain one mathematical function to model the quantitative relationship between two of clinical trial parameters,
wherein said non-linear regression analysis for relationship between N and GSER comprises a step of fitting by using GSER=a·e^{bN}+c, wherein a, b and c are constants to be determined in the non-linear regression analysis for relationship between N and GSER;
wherein said non-linear regression analysis for relationship between N and CTER comprises a step of fitting by using CTER=A·(1−e^{BN})+C, wherein A, B, and C are constants to be determined in the non-linear regression analysis for relationship between N and CTER;
wherein said one or more processors, in view of need or priority of said clinical trial, further extrapolate values of the two of clinical trial parameters and confidence levels of a plurality of data points according to said mathematical function, wherein extrapolated value of N is or is approximated to a natural number; and
(v) an outputting unit that outputs the optimal values for the two clinical trial parameters for said enrollment plan corresponding to the highest confidence level, wherein said optimal values for said clinical trial parameters are used in the design of said enrollment plan for said clinical trial associated with said disease or condition.

13. The system of claim 12, wherein said confidence levels are mapped out on a chart corresponding to said mathematical function as contour lines representing said confidence levels.

14. The system of claim 12, wherein said confidence levels are selected from the group consisting 2 0 of 70%, 80%, 90%, 95%, 98%, and 99%.

15. The system of claim 12, wherein said disease or condition is selected from the group consisting of metabolic disease, respiratory disease, neurologic disease, and cancer.

16. The system of claim 12, wherein the quantitative relationship between N and CTER for clinical trials associated with a single metabolic disease or condition is represented by the formula CTER=363.7·(1−e^{-0.00124N})+5.58.

17. The system of claim 12, wherein the quantitative relationship between N and CTER for clinical trials associated with a single respiratory disease or condition is represented by the formula CTER=204.6·(1−e^{-0.00371N})+3.1.

18. The system of claim 12, wherein the quantitative relationship between N and CTER for clinical trials associated with a single neurologic disease or condition is represented by the formula CTER=37.4·(1−e^{-0.0132N}).

19. The system of claim 12, wherein the quantitative relationship between N and GSER for clinical trials associated with a single metabolic disease or condition is represented by the formula GSER=1.10·e^{-0.0193N}+0.311.

20. The system of claim 12, wherein the quantitative relationship between N and GSER for clinical trials associated with a single respiratory disease or condition is represented by the formula GSER=0.715·e^{-0.00533N}+0.291.

21. The system of claim 12, wherein the quantitative relationship between N and GSER for clinical trials associated with a single neurologic disease or condition is represented by the formula GSER=0.330·e^{-0.00482N}+0.264.

22. A method of evaluating an enrollment proposal for a clinical trial associated with a disease or condition, wherein said method comprises the steps of:
(i) calculating proposed values of one or more clinical trial parameters in said proposal, wherein said one or more clinical trial parameters are selected from the group consisting of number of investigator sites (N), gross site enrollment rate (GSER), and clinical trial enrollment rate (CTER), wherein GSER is defined as the number of patients enrolled at one investigator site in a unit of time, and CTER rate is defined as the number of patients enrolled in a unit of time;
(ii) projecting a data point corresponding to the proposed values of said one or more clinical trial parameters of said proposal, to a chart corresponding to the mathematical function of claim 1; and
(iii) via an outputting unit, outputting a value of confidence level for said proposal.

23. A system for evaluating an enrollment proposal for a clinical trial associated with a disease or condition, said system comprising:
(i) a converting unit that calculates or derives proposed values of one or more clinical trial parameters for said proposal, wherein said one or more clinical trial parameters include but are not limited to number of investigator sites (N), gross site enrollment rate (GSER), and clinical trial enrollment rate (CTER), wherein GSER is defined as the number of patients enrolled at one investigator site in a unit of time, and CTER rate is defined as the number of patients enrolled with a clinical trial in a unit of time;
(ii) a projecting unit for projecting the proposed values of said one or more clinical trial parameters to a chart corresponding to the mathematical function of claim 1; and
(iii) an outputting unit that outputs a value of confidence level for said proposal.
